(19) United States
(12) Patent Application Publication
(10) Pub. No.: US 2022/0112467 A1
(43) Pub. Date: Apr. 14, 2022

(54) CLOSED-SYSTEM METHOD AND KIT OF DISPOSABLE ASSEMBLIES FOR ISOLATING MESENCHYMAL STROMAL CELLS FROM LIPOASPIRATE

(71) Applicant: VBC Holdings LLC, Culver City, CA (US)

(72) Inventors: Giacomo CATTARUZZI, Udine (UD) (IT); Arianna BARCHIESI, Udine (UD) (IT); Alice PAULITTI, Udine (UD) (IT); Mattia SIMEONI, Pagnacco (UD) (IT); Massimo MORETTI, Povoletto (UD) (IT)

(73) Assignee: VBC Holdings LLC, Culver City, CA (US)

(21) Appl. No.: 17/496,269

(22) Filed: Oct. 7, 2021

Related U.S. Application Data
(60) Provisional application No. 63/089,613, filed on Oct. 9, 2020.

(51) Int. Cl. C12N 5/077; C12N 5/00

(52) U.S. Cl. CPC C12N 5/0669; C12N 5/0087; A61M 1/815; A61M 2202/08; C12N 2509/00

(57) ABSTRACT

A closed-system (i.e., hermetically sealed) methods, and kits for isolating Mesenchymal Stromal Cells (MSCs) from lipoaspirate, that utilizes specific hermetically-sealed, disposable assemblies and containers that are aseptically interconnected, are disclosed. Contemplated methods require lipoaspirate as a starting material, and provide for obtaining isolated, purified MSCs at the end of the process. Kits are contemplated to contain disposable assemblies, composed by sterile components such as modified syringes, tubing, centrifuge tubes, filtering units, that can be aseptically connected while maintaining sterility thereby keeping the system closed (i.e., hermetically sealed) with respect to the external environment. As a result, contamination during the isolation process can be avoided. The MSCs that are obtained at the end of the processing are thus ready to be further manipulated in subsequent operations (in example, expansion) also for therapeutic purposes.

### CROSS-REFERENCE TO RELATED APPLICATIONS

This application claims the benefit of priority from U.S. Provisional Application No. 63/089,613, filed on Oct. 9, 2020, which is incorporated by reference herein.

### FIELD OF INVENTION

The field of the invention relates to technologies for purification of cells, more specifically purification of mesenchymal stromal cells (MSCs).

### BACKGROUND

To date, there are several systems and methods for processing adipose tissue and/or lipoaspirate and to extract stem cells. In example, WO2012148502A2 describes a system for lipoaspirate stem cell separation that features a container and a vacuum source; U.S. Pat. No. 9,931,445B2 describes a system and methods for preparing adipose-derived stem cells by means of a single modified centrifuge tube; U.S. Pat. No. 9,453,200B2 describes an apparatus for isolating cells from adipose tissue, that includes a lipid separation processor; U.S. Pat. No. 7,687,059B2 describes a closed-system method for treating patients with processed lipoaspirate cells. Several methods for aseptically connect two termini of tubing exist, described for example in US20110220290A1 where a heat-driven aseptic welding as a way for connecting tubing is used; U.S. Pat. No. 8,448,992B2 describes a method and apparatus for sealing tubing or tubing assemblies; and US20100224329A1 describes another apparatus for connecting two sections of a tubing using a laser source. However, the known systems fail to provide for or yield purified MSCs having desirable properties.

### SUMMARY

The present disclosure relates to a method and kit for isolating MSCs from lipoaspirate, that utilizes a number of disposable assemblies in a closed-system fashion. Stated otherwise, the entire processing occurs without ever allowing the medium or cells coming in contact with the environment. As a result, sterility throughout the process can be maintained. The method requires aseptic fluid communication (i.e., sterile connection and sterile disconnection) of different assemblies in the kit. This is achieved with single-use, disposable, sterile interconnected components in the kit. An embodiment also devises a series of disposable assemblies where reactions take place (such as those where enzymatic dissociation of fat tissue occurs, etc.) and where bags or equivalent containers containing reagents can be connected.

In the present disclosure, the sterile connection and disconnection of the assembles is achieved with an aseptic fluid communication means. One such example of aseptic fluid communication means are welded portions of tubing made from a polymeric material. Aseptic cutting and welding of polymeric tubing portions is a widely known method and is currently a common standard method where aseptic connections of tubing are required. This method, due to common use, is preferable for connecting the different disposable assemblies and bags as compared to other aseptic fluid communication means known in the art.

MSCs isolated from lipoaspirate/adipose tissue (AD-MSCs) are widely used in the field of cell therapy, as their therapeutic potential has been demonstrated by a number of clinical trials and applications, ranging from musculoskeletal, cardiovascular, lung and spinal cord injuries, autoimmune diseases, liver, bone and cartilage diseases, and COVID-19's ARDS (Acute Respiratory Distress Syndrome) among others. MSCs are extracted and purified from lipoaspirate, and can be administered to patients without further manipulation, or after an expansion phase in order to obtain a larger number of cells. MSCs can be frozen and stored for administration to patients in case of future needs.

The present technology provides a closed-system (i.e., hermetically sealed) method and a kit that allows cells isolation from adipose tissue, for subsequent AD-MSCs expansion, in a cleanroom with background area classified as of grade D (100,000) as per Aseptic Manufacturing Principles of the Good Manufacturing Practice (GMP) specific to Advanced Therapy Medicinal Products (ATMPs), because the process of isolation occurs without opening the system to the external environment due to be hermetically sealed. The resulting captured or purified cell population have superior properties, growth, or expansion properties than has been previously achieved.

### BRIEF DESCRIPTION OF THE INVENTION

An object of the inventive subject matter is to provide a method and a kit for isolating mesenchymal stem cells from a lipoaspirate sample that is obtained from a patient, through the use of dedicated disposable assemblies and reagents contained in different bags. Lipoaspirate is initially collected in a modified syringe, which is subsequently welded to a first disposable assembly and thereby isolating the sample from the environment. Following welding, the sample is initially deprived from red blood cells (RBCs) which are transferred to a dedicated waste bag along a dedicated tubing line, while again ensuring the sample remains isolated. Subsequently, RBCs-deprived lipoaspirate is transferred in a tube, while a bag containing an enzymatic agent is welded to the same tubing line, so that the digestion of lipoaspirate occurs within the tube. Digestion is typically carried out at 37° C., with periodic agitation of the mixture. Following digestion, a bag containing a quenching agent is welded to the same tubing line, and quenching agent is transferred in the same tube. The first disposable assembly is then welded to a second, different, disposable assembly, and the mixture is filtered through a filtering unit and collected in a centrifuge tube. The filtering unit is removed from the tube and the tube can then centrifuged in order to separate the cells and supernatant. Supernatant is then transferred to a waste bag which was previously welded, then a bag containing a resuspension reagent is welded to the same tubing line, and an appropriate volume of reagent is transferred to the tube, where the pelleted cells are resuspended. Finally, a modified syringe can be welded to sample a volume of resuspended cells (in example, to count, characterize or examine cells off-line), and another modified syringe is welded in order to obtain the isolated cells. These can be subsequently transferred, by welding appropriate containers along a dedicated tubing line, in example to seed and expand them in automated expansion systems, or to other devices for other applications.

In one embodiment, provided is a closed-system method for isolating mesenchymal stromal cells from lipoaspirate obtained from a patient. The method includes the following steps:

i) providing a sample of lipoaspirate from the patient;

ii) removing red blood cells from the lipoaspirate sample from step i);

iii) enzymatically digesting the lipoaspirate sample from step ii) after removal of the red blood cells;

iv) quenching the enzymatic digestion of the lipoaspirate sample from step iii);

v) filtering the enzymatically-digested, lipoaspirate sample from step iv);

vi) separating the mesenchymal stromal cells from the supernatant in the filtered, lipoaspirate sample from step v);

vii) resuspending the separated mesenchymal stromal cells from step vi) with cell culture medium; and

viii) evaluating the resuspended mesenchymal stromal cells from step vii) for cell expansion;

wherein all of steps are conducted under aseptic conditions to avoid contact with the external environment.

In an additional embodiment, the method further includes step ix) expanding the resuspended cells from step vii) under aseptic conditions. In another embodiment, the step of expanding the resuspended cell is carried out under manual conditions. In another embodiment, the step of expanding the resuspended cell is carried out with a bioreactor, such as an automated bioreactor.

In yet another embodiment, provided is a disposable kit for isolating mesenchymal stromal cells from lipoaspirate from a patient, the kit comprising the following components:

a syringe configured to obtain a lipoaspirate sample from the patient,

a red blood cell removal assembly being configured to remove red blood cells from the lipoaspirate sample under aseptic conditions and configured to leave behind a red blood cell deprived lipoaspirate sample;

an enzymatic digestion assembly configured to transfer an enzymatic solution to the red blood cell assembly under aseptic conditions;

a quenching assembly configured to transfer a quenching solution to red blood cell assembly under aseptic conditions;

a filtering assembly configured to receive and filter the digested lipoaspirate sample from the red blood assembly under aseptic conditions, the filtering assembly being further configured for insertion into and extraction from a laboratory centrifuge;

a waste assembly configured to receive supernatant from the filtering assembly un aseptic conditions;

a cell culture medium assembly configured to transfer cell culture medium to the filtering assembly under aseptic conditions to resuspend mesenchymal stromal cells from the lipoaspirate sample; and

a sample syringe configured to receive a sample of the resuspended mesenchymal stromal cells from the filtering assembly under aseptic conditions;

wherein each of the components are configured with one or more aseptic fluid communication means to transfer, receive, or store material under aseptic conditions.

In another embodiment, the kit further includes a storage syringe configured to receive under aseptic conditions the resuspended mesenchymal stromal cells from the filtering assembly, the storage syringe being further configured to store and transfer the resuspended mesenchymal stromal cells under aseptic conditions due to the one or more aseptic fluid communication means.

In yet another embodiment, the kit further includes a storage assembly configured to receive and store under aseptic conditions the resuspended mesenchymal stromal cells from either the filtering assembly or the storage syringe due to one or more aseptic fluid communications means. An example of one or more aseptic fluid communications means is aseptic weldable tubing, such as aseptic weldable tubing formed a thermoplastic polymer.

### BRIEF DESCRIPTION OF THE DRAWINGS

FIG. 1 is a modified syringe that is used to initially contain the lipoaspirate sample obtained by the patient;

FIGS. 2A, 2B and 2C are disposable assemblies where the first steps of isolation occur;

FIGS. 3A, 3B and 3C are disposable assemblies where subsequent steps of isolation occur;

FIG. 4 is a modified syringe that is used for sampling cells to allow subsequent off-line analysis;

FIG. 5A is a modified syringe for collecting isolated cells and for eventually preparing them for subsequent seeding in an expansion system.

FIG. 5B is a modified bag where cells can be mixed with cell culture medium.

FIG. 6 is a flow diagram illustrating the steps for isolating cells in accordance with an embodiment.

FIG. 7 is a photomicrograph showing MSCs at one (1) day-post seeding.

FIG. 8 is a photomicrograph showing MSCs at three (3) days post-seeding.

FIG. 9 is a photomicrograph showing MSCs at six (6) days post-seeding (harvesting day).

### DETAILED DESCRIPTION OF THE INVENTION

Hereunder is a description of a closed-system (i.e., hermetically sealed) method and kit for isolating mesenchymal stem cells (MSCs) from a lipoaspirate sample that is obtained from a patient, by means of dedicated disposable assemblies with aseptic fluid communication means. Stated otherwise, the dedicated disposable assemblies are all configured to transfer, receive, or store the required materials under aseptic conditions. This is achieved, in part, from each assembly being adapted with one or more aseptic fluid communication means, which are described in further detail below.

The dedicated disposable assemblies (e.g., bags and related tubing) are typically formed from a thermoplastic polymer, which is flexible at room temperature. In contrast, centrifuge tubes used in the assemblies are typically formed from a polymer that is rigid at room temperature. In accordance with an embodiment, the thermoplastic polymer can be formed from a single monomer (e.g., ethylene or propylene) or from co-monomers (e.g., ethylene with a hexane co-monomer in the chain). A benefit of thermoplastic polymers is that the material can be easily cut, and heat welded to provide an aseptic fluid communication means (e.g., a hermetically sealed connection) between multiple assemblies. In addition, individual assembles can be cut and welded multiple times due to the benefit of tubing made from thermoplastic polymers. Other aseptic fluid communications means can be utilized in accordance with an embodiment such as aseptic quick connect couplers. Although due to the prevalence of welding equipment, cutting/welding thermoplastic tubing to connect the assemblies is preferred.

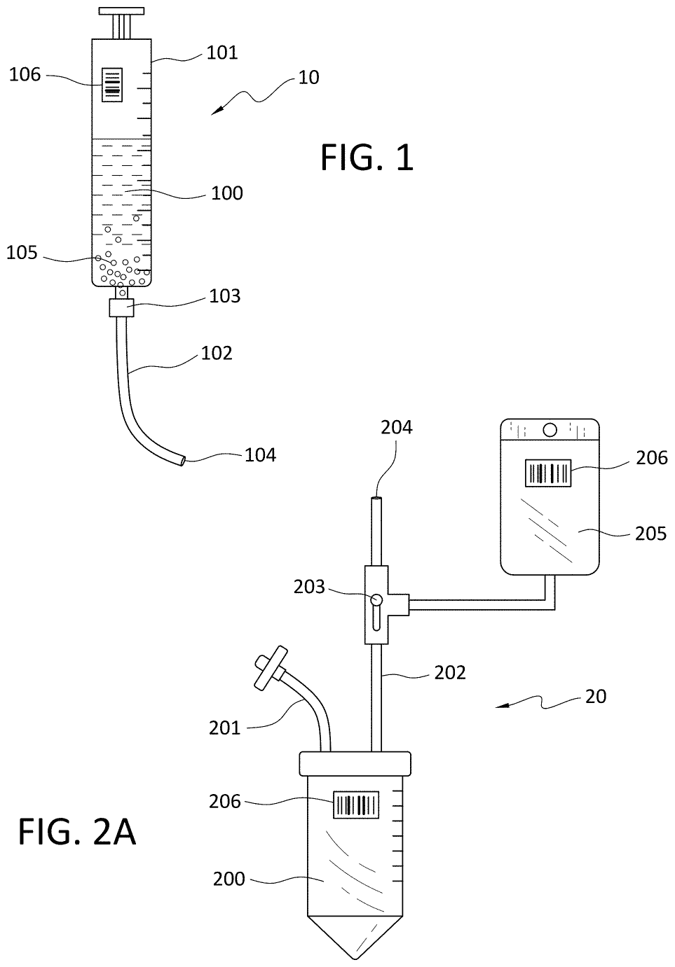

With reference to FIG. 1, an embodiment of disposable assembly 10 is shown as lipoaspirate sample 100 collected from a patient with a modified syringe 101. Syringe 101 is in fluid communication with a dead-end tubing 102 attached to the barrel tip 103 of syringe 101. Dead-end tubing 102 is adapted with a hermetically sealed terminus 104 at the end opposite from syringe 101 to maintain a closed system. Typically, the lipoaspirate is left standing in the syringe for 15 minutes at room temperature, or 5 minutes at 37° C., to allow red blood cells 105 form a layer at the bottom of the suspension due to gravity.

As shown in FIG. 1, syringe 101 is also provided with label 106 to allow tracking of the sample throughout the isolation process. Label 106 can be a barcode, a RFID label, or any other equivalent means to track the sample throughout the isolation process. More sophisticated tracking and sample verification methodologies can also be used. One such methodology is described in co-pending, U.S. application Ser. No. 16/561,773 entitled “Production and Delivery Tracking and Sample Verification of Patient-Specific Therapeutics” assigned to Nantcell, Inc., and is incorporated herein by reference. Another such methodology is described in PCT Publication No.: WO2018/057520 entitled “Sample Tracking Via Sample Tracking Chains, Systems and Methods” assigned to Nant Holdings IP, LLC and is also incorporated herein by reference.

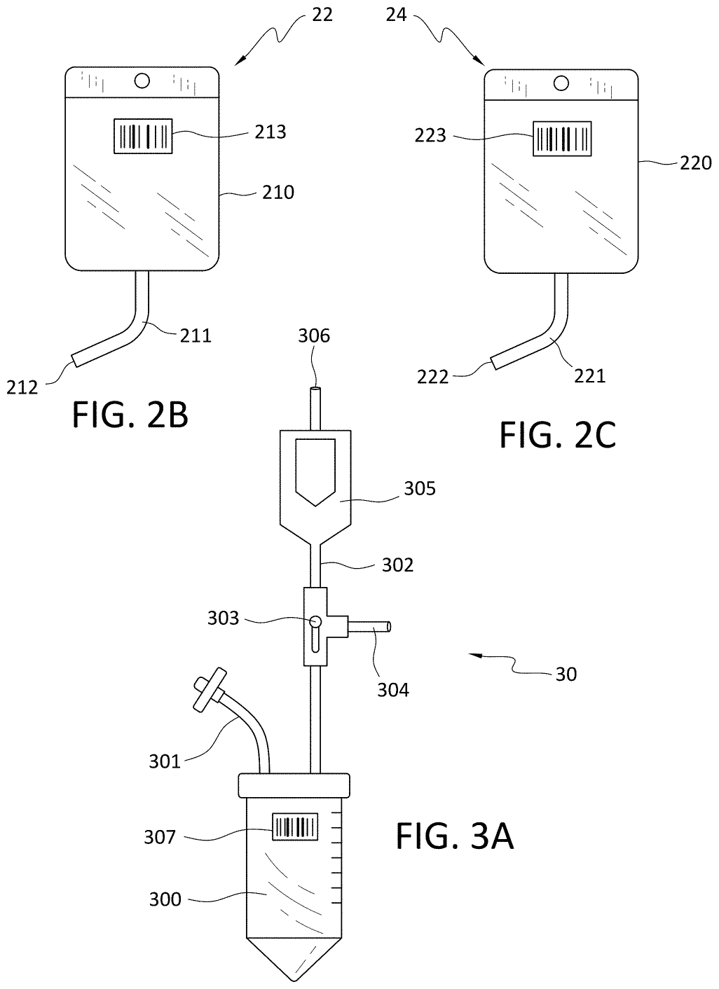

With reference to FIG. 2A, an embodiment of disposable assembly 20 is shown as centrifuge tube 200 modified with a cap that features two different tubing lines in fluid communication with interior of centrifuge tube 200. While a centrifuge tube is used in assembly of 20 of FIG. 2A, other equivalent containers can also be used as long the containers are configured for insertion and extraction from a centrifuge. Stated otherwise, the container should have shape and size that allows for insertion and extraction from a centrifuge.

Turning back to FIG. 2A, the first tubing line 201 includes a 0.2 μm filter at its end opposite from centrifuge tube 200. The second tube 202 is adapted with a stopcock 203 to selectively provide fluid communication with centrifuge tube 200 or bag 205 but not both at the same time. Stopcock 203 also is adapted with a dead-end tubing with a hermetically sealed terminus 204 on the side stopcock 203 opposite from centrifuge tube 200. One skilled in the art will also appreciate that any suitable aseptic valve means can be used as a substitute for the stopcock. The use of a stopcock is merely an example of an aseptic valve means. As further shown in FIG. 2A, both centrifuge tub 200 and bag 205 are equipped with label 206 to allow tracking and authentication during the process of isolating the cells.

In accordance with an embodiment, termini 104 of assembly 10 and 204 of assembly 20 are connected (i.e., placed in fluid communication with each other), for example, by cutting-and-welding. By turning the stopcock 203, the red blood cells (RBCs) layer is transferred from syringe 101 to the bag 205 by a technician applying force to the syringe plunger (not labelled). In another embodiment, termini 104 and 204 can feature an aseptic fluid communication means (e.g., aseptic connector) so that fluid communication occurs while avoiding contact with the external environment. Once the RBC layer is transferred, the technician then switches stopcock 203 to open tubing line 202 and to close access to bag 205. Once tubing line 202 is opened, the RBCs-deprived lipoaspirate is transferred from syringe 101 to the centrifuge tube 200 by the technician. As noted above, centrifuge tube 200 can substituted by a bag or by another closed (i.e., hermetically sealed) container. Either of which will provide temporary aseptic storage for the lipoaspirate.

With reference to FIG. 2B, an embodiment of disposable assembly 22 is shown as bag 210 containing an enzymatic solution that digests adipose tissue contained in the lipoaspirate. One such enzyme to be used for digestion is collagenase type II. To provide aseptic fluid communication with tube 200, bag 210 is adapted with a tubing line 211 having a hermetically sealed terminus 212 at the end of the tube opposite from bag 210. For enzymatic digestion to occur, a technician cuts the tubing line to bag 205 and discards bag 205. Tubing line 202 is then connected to tubing line 211 (in example, by cutting-and-welding) so that the enzymatic solution can be transferred to the tube 200 again without exposure to the external environment. The mixture is allowed to incubate at 37° C. with periodic agitation to facilitate enzymatic digestion of fat tissue in the sample. Also shown in FIG. 2B is that bag 210 is provided with label 213 to allow tracking and authentication during the process of isolating the cells.

Periodic agitation can be achieved by any conventional technique. For example, the bag can be placed on a rocker or a tilt/shake mechanism to agitate the sample. The tilt/shake mechanism can also be adaptive and react to data recorded by sensors (e.g., microscope, accelerometers, scales, etc.) on the tilt/shake mechanism. One such adaptive tilt/shake mechanism is described in U.S. Pat. No. 10,801,005 entitled “Systems, Apparatus and Methods for Controlling a Movement of a Cell Culture to Optimize Cell Growth”, which is assigned to VivaBioCell Holdings, LLC and is incorporated herein by reference. U.S. Pat. No. 10,801,005 describes a system including a tray configured to hold a container containing a cell culture where the movement of the tray is adjusted based on a camera image of the cell culture. With the appropriate algorithm, the processor of the tilt/shake mechanism can determine the progress of enzymatic digestion. Based on the progress, the processor will then adjust the rate of movement based on one or more sensor inputs. The tilt/shake mechanism can also include a variety of sensors to monitor other parameters of the cell culture. These are discussed in more detail in the above-cited patent.

With reference to FIG. 2C, an embodiment of disposable assembly 24 is shown as bag 220 containing a quenching solution that blocks (i.e., stops) the action of the enzyme. Examples of quenching solutions to be used include, but are not limited to, phosphate buffered saline (PBS) and bovine serum albumin (BSA). Bag 220 features a tubing line 221 with a hermetically sealed terminus 222 at the end opposite from the bag. A technician then cuts tubing line 202 and discards bag 210. Tubing lines 221 and 202 are then connected (in example, by cutting-and-welding) so that the quenching solution can be transferred to the centrifuge tube 200. Also shown in FIG. 2C is assembly 24 provided as bag 220 with label 223 to allow tracking and authentication during the process of isolating the cells. After a sufficient amount of time has passed, bag 220 can be discarded as further described below.

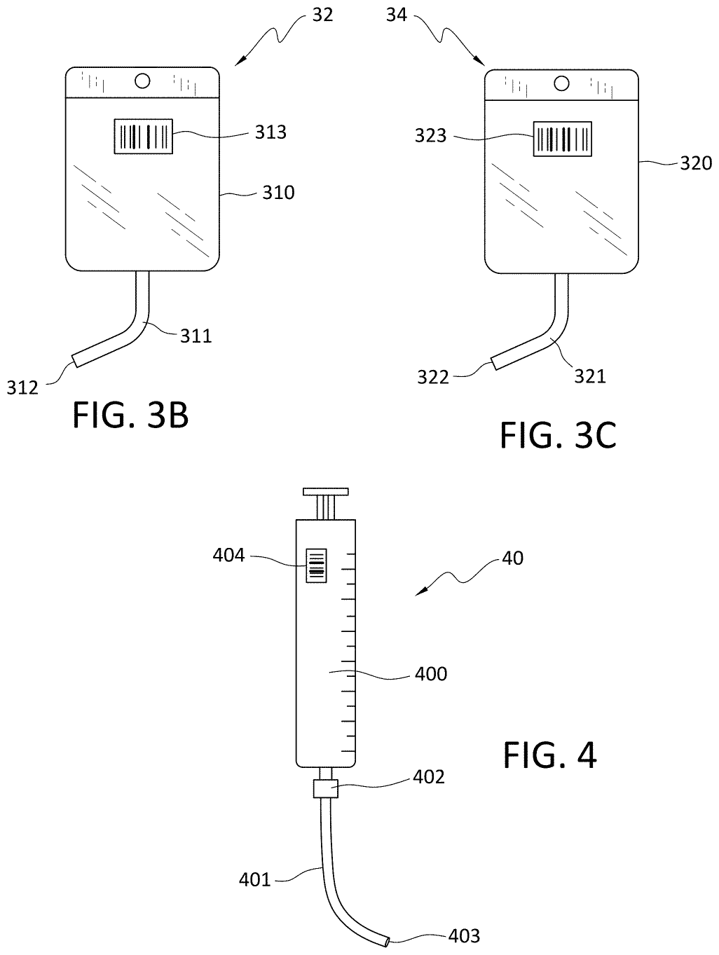

With reference to FIG. 3A, an embodiment of disposable assembly 30 is shown as centrifuge tube 300 modified with a cap that features two different tubing lines in a similar fashion to centrifuge tube 200 of assembly 20 but with some modifications. The cap has a first tubing line 301 with a 0.2 μm filter at its end opposite from centrifuge tube 300. The cap also has a second tubing 302 that includes a stopcock 303, a dead-end tubing line 304 extending from stopcock 303, and a filtering unit 305 on the side of stopcock 303 opposite from centrifuge tube 300. Filtering unit 305 also has a dead-end terminus 306 on the side opposite from stopcock 303. Dead-end terminus 306 is hermetically sealed like all the other termini in accordance with an embodiment. Also shown in FIG. 3A is that centrifuge tube 300 is provided with label 307 to allow tracking and authentication during the process of isolating the cells.

Pursuant to the isolation process, a technician cuts tubing line 202 and discards bag 220. Tubing line 202 of assembly 20 and terminus 306 of assembly 30 are thereafter connected (in example, by cutting-and-welding) to provide an aseptic fluid communication means thereby allowing the mixture (i.e., cell suspension) to be transferred to the tube 300. Filtering unit 305 filters the mixture as the mixture is transferred from centrifuge tube 200 to centrifuge tube 300. In accordance with an embodiment, the transferring of material is simply achieved with the use of gravity. However, material transfer can also be assisted with pumps that do not alter the closed systems. One such pump is a peristaltic pump. After transfer, centrifuge tube 200 is then discarded by the technician.

In one embodiment, filtering unit 305 includes two different filtering barriers, with pore diameters of 175 μm and 40 μm, respectively. Filtering allows to separation of adipose tissue and fibrous tissue (retained in the filtering unit 305) from the rest of the suspension that is collected in the tube 300. After transfer to tube 300, tubing line 302 is cut and hermetically sealed (in example, by cutting-and-welding) in the region between the stopcock 303 and the filtering unit 305. The tube 300 is then centrifuged (in example at 500xg for 5 minutes) in order to pellet the cells contained in the stromal vascular fraction (SVF).

With reference to FIG. 3B, an embodiment of disposable assembly 32 is shown as empty bag 310 featuring a tubing line 311 with a hermetically sealed terminus 312 at the end of tubing line 311 opposite from bag 310. Tubing lines 311 of assembly 32 and 302 of assembly 30 are connected (in example, by cutting-and-welding) so that the supernatant, obtained in the tube 300 after centrifugation, is transferred to the waste bag 310 to be discarded. After transfer, the pelleted cells are left behind in centrifuge tube 300. Once again, transfer is simply achieved with the use of gravity. However, pumps that do not affect the hermetic seal can also be used. Also shown in FIG. 3B is that bag 310 can be provided with label 313 to allow tracking and authentication during the process of isolating the cells.

With reference to FIG. 3C, an embodiment of disposable assembly 34 is shown as bag 320 containing a cell culture medium for resuspending the pelleted cells. Bag 320 features a tubing line 321 with a hermetically sealed terminus 322 at the end of tubing line 321 opposite from bag 320. Tubing lines 321 and 302 are connected (in example, by cutting-and-welding) so that the cell culture medium can be transferred to the tube 300. Here, the pellet containing cells is resuspended by gentle flicking/agitation of tube 300 by the technician. Also shown in FIG. 3C is that bag 320 can be provided with label 323 to allow tracking and authentication during the process of isolating the cells.

With reference to FIG. 4, an embodiment of disposable assembly 40 is shown as modified syringe 400 featuring a dead-end tubing 401 in fluid communication with barrel tip 402 of syringe 400. Dead-end tubing 401 is adapted with a hermetically sealed terminus 403 at the end opposite from syringe 400 to maintain a closed system. Tubing lines 401 of assembly 40 and 304 of assembly 30 can be connected (in example, by cutting-and-welding) to provide an aseptic fluid communication means so that an amount of the resuspended cell suspension can be sampled with the modified syringe. For example, the sample can be optionally subjected to off-line analysis such as cell count, cell density, immunophenotyping, viability evaluation, and the like. The results of the sample analysis can also be tracked via use of one or more notarized or distributed ledger technologies as previously indicated. Also shown in FIG. 4 is that syringe 400 can be provided with label 404 to allow tracking and authentication during the process of isolating the cells.

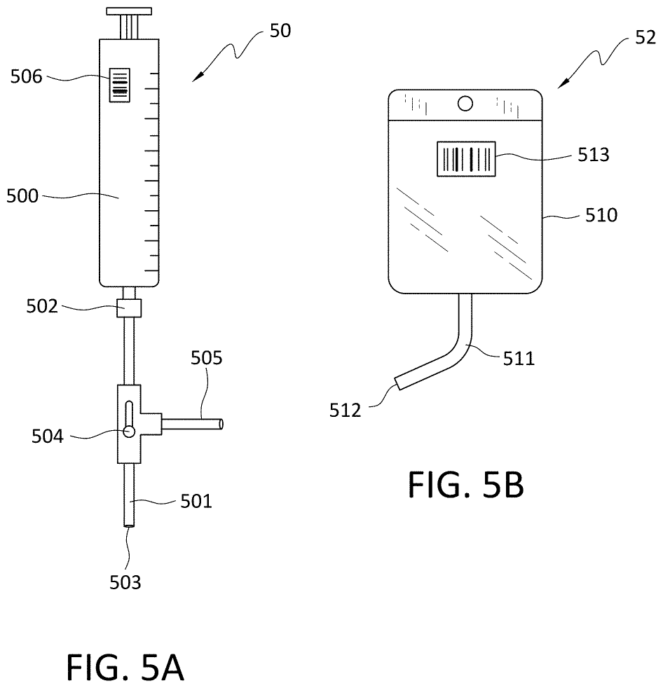

With reference to FIG. 5A, an embodiment of disposable assembly 50 is shown as modified syringe 500. Syringe 500 includes a dead-end tubing 501 in fluid communication with the barrel tip 502 of syringe 500. Dead-end tubing 501 also has a hermetically sealed terminus 503 opposite from barrel tip 502 and a stopcock 504 positioned between terminus 503 and stopcock 504. Stopcock 504 also has a dead-end tubing line 505 extending therefrom. Stopcock 504 is configured to selectively open and close access to tubing lines 501 and 505. To remove the resuspended cells from centrifuge tube 300, tubing lines 501 and 304 are connected (in example, by cutting-and-welding) to provide an aseptic fluid communication means so that the resuspended cell suspension can be transferred to the syringe 500. After which, the technician discards centrifuge tube 300. Tubing line 501 is hermitically sealed (in example, by cutting-and-welding) so that the syringe 500, containing the cell suspension, can be shipped, stored, or used as an input for subsequent manipulations. Also shown in FIG. 5A is that syringe 500 is provided with label 506 to allow tracking and authentication during the process of isolating the cells.

With reference to FIG. 5B, an embodiment of disposable assembly 52 is shown as bag 510 containing cell culture medium allowing for subsequent expansion of the cultured cells using a bioreactor. Bag 510 features a tubing line 511 with a hermetically sealed terminus 512 at the end of tubing line 511 opposite from bag 520. Transfer of the resuspended cells is achieved by connection tubing line 511 and 505 (in example, by cutting-and-welding). Once connected, stopcock 504 is turned to open access to tubing 505 and a technician presses the plunger (not labelled) of syringe 500 to transfer the cell suspension to the bag 510 and is mixed with the cell culture medium. In an embodiment, the tubing line 511 can be hermetically sealed (in example, by cutting-and-welding). Bag 510, now containing the resuspended cells, can now be connected (in example, by using tubing line 511 and cutting-and-welding) to any other tubing where a system for cell expansion (automatic or manual) can import the content of the bag.

One such system for automatic cell expansion is the Nant XL automated bioreactor system sold by VivaBioCell S.p.A., located in Udine, Italy. Bag 510 can be placed into the Nant XL bioreactor for expansion. In the alternative, the resuspended cells can be directly transferred to the Nant XL cell culture flask using syringe 500. Both bag 510 and the Nant XL cell culture flask are then provided with aseptic fluid communication means to the various components of the bioreactor. After which, cell expansion is allowed to occur for a sufficient amount of time until the required parameters are meet for successful expansion.

Another such system for automatic cell expansion is described in US Publication No.: US2017/0037357 entitled “Automated Cell Culturing and Harvesting Device”, which is also assigned to VivaBioCell S.p.A. and incorporated herein by reference. Using syringe 500, the resuspended cells can be directly transferred to the multilayered, cell culture chamber described in US2017/0037357. After which, cell expansion is allowed to occur for a sufficient amount of time until the required parameters are meet for successful expansion.

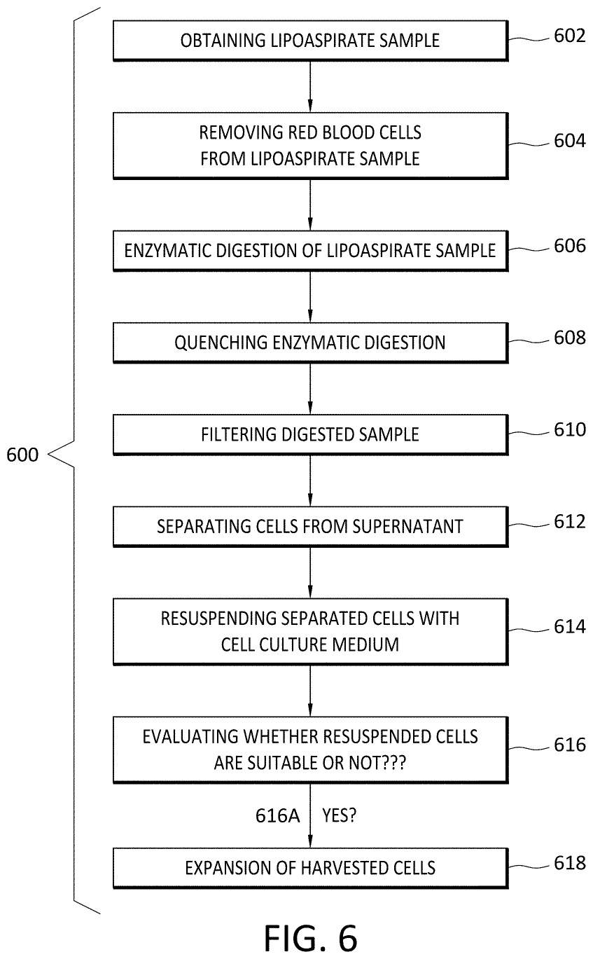

With reference to FIG. 6, provided is a flow diagram describing the general steps of closed-system method 600 of an embodiment of the invention. In one embodiment, a sample of lipoaspirate is first obtained from a patient (step 602) using syringe 100 from the kit as described above. Step 602 also includes hermetically sealing dead-end tubing 102 to form terminus 104 to maintain a closed system as discussed above.

Thereafter, the method entails removing the red blood cells (RBCs) from the lipoaspirate sample (step 604) once the RBCs are allowed to settle to the bottom of syringe 100. In one embodiment, step 604 is achieved using centrifuge tube 200 and bag 205 from the kit which are in aseptic fluid communication with each other. After the RBCs are transferred to bag 205 from syringe 101, the RBCs-deprived lipoaspirate is transferred from syringe 101 to the centrifuge tube 200 after stopcock 203 is actuated to allow open access to centrifuge tube 200. Bag 205 is discarded by the technician.

As further shown in FIG. 6, the RBCs-deprived lipoaspirate is subjected to enzymatic digestion (step 606). In one embodiment, step 606 is achieved using centrifuge tube 200 and bag 210 containing the enzymatic solution from the kit as discussed above. Transfer of the enzymatic solution (not labelled) is achieved by placing centrifuge tube 200 and bag 210 in aseptic fluid communication with each other. This can be achieved by cutting and welding together tubing line 202 and tubing line 211 so that the enzymatic solution can be transferred to the tube 200. Once the enzymatic solution has been transferred, a technician can cut and discard bag 210.

After a sufficient amount of time has passed, the enzymatic reaction in centrifuge tube 200 must be quenched (i.e., stopped) (step 608). In one embodiment, step 608 is achieved using centrifuge tube 200 and bag 220 containing the quenching solution from the kit. Transfer of the quenching solution is achieved by placing centrifuge tube 200 and bag 220 in aseptic fluid communication with each other. This can be achieved by cutting and welding together tubing line 202 and tubing line 221 so that the quenching solution can be transferred to the tube 200. After a sufficient amount of time, a technician can cut and discard bag 220.

Once the enzymatic reaction has been quenched, the digested lipoaspirate sample is filtered (step 610). In one embodiment, step 610 is achieved using centrifuge tube 200 and centrifuge tube 300 from the kit. As described above, centrifuge tube 300 is modified with a cap that features two different tubing lines in a similar fashion to centrifuge tube 200 but with some additional modifications. Centrifuge tube 300 includes filtering unit 305 with dead-end terminus 306. Dead-end terminus 306 is hermetically sealed like all the other termini in accordance with an embodiment. Transfer and filtering of the mixture us achieved by placing centrifuge tubes 200 and 300 in aseptic fluid communication with each other. This can be achieved by cutting and welding together tubing line 202 and terminus 306 to effect transfer and filtering. Once the mixture is filtered, a technician can cut and discard centrifuge tube 200.

Once filtering is completed, the MSCs are separated from the cell medium (step 612) in centrifuge tube 300. In one embodiment, this is achieved by first removing filtering unit 305 from the assembly. A technician cuts and hermetically seals tubing line 302 located between stopcock 303 and filtering unit 305. Filtering unit 305 can then be discarded. Centrifuge tube 300 and stopcock 303 are then placed in a conventional laboratory centrifuge where the assembly is spun to form a pellet from the cells and a supernatant from the cell medium. Step 612 also includes the removal of the supernatant from tube 300. This is achieved by placing empty waste bag 310 from the kit in aseptic fluid communication with tube 300. A technician then hermetically seals tubing line 311 of waste bag 310 to remaining tubing line 302 connected to stopcock 303. After the supernatant is transferred to waste bag 310, waste bag 310 can be discarded by the technician leaving behind the pelleted cells in centrifuge tube 300. It is also contemplated that the cells can be isolated via mechanical technique as described in WO 2019/140104 titled “Microfluidic Cellular Device and Methods of Use Thereof”, filed Jan. 10, 2019.

Once separation of the pellet is completed, the cells are resuspended with fresh cell culture medium (step 614). This is achieved by cutting and hermetically sealing tubing lines 321 and 302 to each other thereby provide an aseptic fluid communication means so that the cell culture medium (not labelled) can be transferred to tube 300 from bag 320 of the kit. The pellet containing the cells is resuspended by gentle flickering/agitation of tube 300 by a technician. Once transfer is completed, cell medium bag 310 can be cut from the assembly and discarded while tubing line can be sealed until further use.

Once the cells are resuspended, a sample of the resuspended are taken to be evaluated whether or not the cells are in condition for further expansion (step 616). This is achieved with the use of modified syringe 400 from the kit to take a sample of resuspended cells. Dead-end tubing 401 of syringe 400 is hermitically sealed and placed in aseptic fluid communication with tubing line 304 from centrifuge tube 300.

If the cells are in condition for expansion, the remaining cells in tubing 300 are removed (step 616A) for further expansion (step 618) in a bioreactor (automated or manual) as discussed above. The remaining cells may be removed from tubing 300 using modified syringe 500 of the kit. Syringe 500 has dead-end tubing line 501, which can be cut and hermitically sealed to tubing line 304 of tube 300 thereby providing an aseptic fluid communication between the two assembles. Once the cells are transferred, tubing line 304 can be cut and tube 300 discarded. Tubing line 501 is hermitically sealed (in example, by cutting-and-welding) so that the syringe 500 can be shipped, stored, or used as an input for subsequent manipulations.

If so desired, the cells stored in syringe 500 can be further transferred to bag 510 from the kit, which contains cell culture medium allowing for subsequent expansion of the cultured cells using a bioreactor (step 618).

### EXAMPLES

The following non-limiting examples illustrate the use of an embodiment to isolate MSCs from the lipoaspirate of a patient.

### Example 1

SVF cells were isolated from fresh adipose tissue using the aseptic, disposable assemblies described above and following the methodology described in FIG. 6. Aseptic conditions were maintained by using weldable tubing as the aseptic fluid communication means. The tubing of each assembly was cut and welded as needed to carry of the method of an embodiment. At the end of the end isolation process, the cells were counted and then expanded under manual conditions. The expanded cells were then characterized through fluorescent activated cell sorting (FACS) analysis.

After 4 isolations with this system, a mean of 15.1 million of cells (SVF fraction; n=4) was obtained starting just from 10 ml of adipose tissue for each isolation from different patients. The SVF fraction was cultivated in aMEM 4F medium (containing 10% FBS, 1% Pen/Strep and four growth factors). The aMEM, FBS and Pen/Strep were obtained from Gibco while the four growth factors were obtained from Sigma Aldrich. After a mean of 6 days of culture (no passages in a new flask), a mean of 12 million of cells (flask T175; n=4) was obtained. The cells were also characterized for MSCs markers and viability with FACS analysis (mean: n=4) that were in line with the typical cultures from adipose tissue. The results are shown in table 1 below.

TABLE 1

| Viability | CD45 | CD14 | CD31 | HLA-DR | CD90 | CD166 | CD73 | CD105 |
|---|---|---|---|---|---|---|---|---|
| 94.70% | 0.89% | 1.79% | 3.22% | 0.09% | 97.28% | 77.06% | 99.67% | 89.19% |

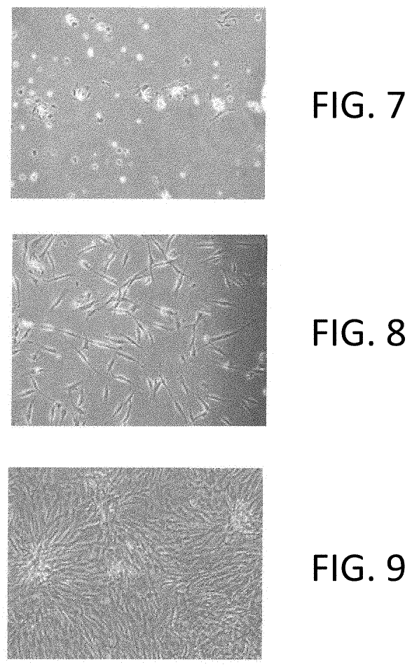

Based on observations, the cells cultivated and expanded in manual conditions presented the correct morphology for MSCs type of cell. The cellular morphology of the isolated and expended MSCs at various stages can be seen in the photomicrographs of FIGS. 7-9. FIG. 7 shows the MSCs at one (1) day-post seeding. FIG. 8 shows the MSCs at three (3) days post-seeding. FIG. 9 shows the MSCs at six (6) days post-seeding at which time the cells were harvested.

What is claimed is:

1. A closed-system method for isolating mesenchymal stromal cells from lipoaspirate obtained from a patient, the method comprises the following steps:
i) providing a sample of lipoaspirate from the patient;
ii) removing red blood cells from the lipoaspirate sample from step i);
iii) enzymatically digesting the lipoaspirate sample from step ii) after removal of the red blood cells;
iv) quenching the enzymatic digestion of the lipoaspirate sample from step iii);
v) filtering the enzymatically-digested, lipoaspirate sample from step iv);
vi) separating the mesenchymal stromal cells from the supernatant in the filtered, lipoaspirate sample from step v);
vii) resuspending the separated mesenchymal stromal cells from step vi) with cell culture medium; and
viii) evaluating the resuspended mesenchymal stromal cells from step vii) for cell expansion;
wherein all of steps are conducted under aseptic conditions to avoid contact with the external environment.

2. The method of claim 1, which further comprises:
ix) expanding the resuspended cells from step vii) under aseptic conditions.

3. The method of claim 2, wherein the step of expanding the resuspended cell is carried out under manual conditions.

4. The method of claim 2, wherein the step of expanding the resuspended cell is carried out with a bioreactor.

5. The method of claim 4, wherein the bioreactor is an automated bioreactor.

6. A disposable kit for isolating mesenchymal stromal cells from lipoaspirate from a patient, the kit comprising the following components:
a syringe configured to obtain a lipoaspirate sample from the patient,
a red blood cell removal assembly being configured to remove red blood cells from the lipoaspirate sample under aseptic conditions and configured to leave behind a red blood cell deprived lipoaspirate sample;
an enzymatic digestion assembly configured to transfer an enzymatic solution to the red blood cell assembly under aseptic conditions;
a quenching assembly configured to transfer a quenching solution to red blood cell assembly under aseptic conditions;
a filtering assembly configured to receive and filter the digested lipoaspirate sample from the red blood assembly under aseptic conditions, the filtering assembly being further configured for insertion into and extraction from a laboratory centrifuge;
a waste assembly configured to receive supernatant from the filtering assembly un aseptic conditions;
a cell culture medium assembly configured to transfer cell culture medium to the filtering assembly under aseptic conditions to resuspend mesenchymal stromal cells from the lipoaspirate sample; and
a sample syringe configured to receive a sample of the resuspended mesenchymal stromal cells from the filtering assembly under aseptic conditions;
wherein each of the components are configured with one or more aseptic fluid communication means to transfer, receive, or store material under aseptic conditions.

7. The disposable kit of claim 6, wherein the kit further comprises a storage syringe configured to receive under aseptic conditions the resuspended mesenchymal stromal cells from the filtering assembly, the storage syringe being further configured to store and transfer the resuspended mesenchymal stromal cells under aseptic conditions due to the one or more aseptic fluid communication means.

8. The disposable kit of claim 6, wherein the kit further comprises a storage assembly configured to receive and store under aseptic conditions the resuspended mesenchymal stromal cells from either the filtering assembly or the storage syringe due to one or more aseptic fluid communications means.

9. The disposable kit of claim 6, wherein the one or more aseptic fluid communications means each comprises aseptic weldable tubing.

10. The disposable kit of claim 9, wherein the aseptic weldable tubing comprises a thermoplastic polymer.
